(19) United States
(12) Patent Application Publication
(10) Pub. No.: US 2006/0246462 A1
(43) Pub. Date: Nov. 2, 2006

(54) Compositions for DNA amplification, synthesis, and mutagenesis

(75) Inventors: Holly Hurlbut Hogrefe, San Diego, CA (US); Michael C. Borns, Escondido, CA (US); Michael L. Muhich, Olivenhain, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Stratagene California

(21) Appl. No.: 11/067,304

(22) Filed: Feb. 25, 2005

Related U.S. Application Data
(63) Continuation of application No. 10/456,848, filed on Jun. 6, 2003, now abandoned.
Application No. 10/456,848 is a continuation of application No. 09/414,295, filed on Oct. 6, 1999, now abandoned.

(51) Int. Cl. C12Q 1/68; C12P 19/34; C12N 9/22

(52) U.S. Cl. 435/6; 435/199; 435/91.2

(57) ABSTRACT

This invention provides compositions comprising a thermostable non-proofreading DNA polymerase, a thermostable proofreading DNA polymerase, and a factor that substantially inhibits the incorporation of undesired nucleotides or analogs thereof into a DNA polymer. The compositions may further comprise a buffer that enhances a polymerization reaction involving DNA polymerases. The invention also provides various methods of amplifying, synthesizing, or mutagenizing nucleic acids of interest using these novel compositions. Kits that comprise the compositions are also provided for amplifying, synthesizing, and mutagenizing nucleic acids.

The following patent applications and patent are hereby specifically incorporated by reference herein: U.S. patent application Ser. No. 08/957,709, filed Oct. 24, 1997; U.S. patent application Ser. No. 08/822,774, filed Mar. 21, 1997; International Application No. PCT/US98/05497, filed Mar. 20, 1998, published as International Publication No. WO 98/42860 on Oct. 1, 1998; U.S. Provisional Patent Application Ser. No. 60/146,580, filed Jul. 30, 1999; U.S. patent application Ser. No. 08/164,290, filed Dec. 8, 1993; U.S. patent application Ser. No. 08/197,791, filed Feb. 16, 1994, which issued as U.S. Pat. No. 5,556,772 on Sep. 17, 1996; U.S. patent application Ser. No. 08/529,767, filed on Sep. 18, 1995.

### BACKGROUND AND SUMMARY OF THE INVENTION

The invention relates to the field of amplifying, synthesizing, and mutagenizing nucleic acids. Further, this invention relates to novel compositions and buffers for amplifying, synthesizing, or mutagenizing nucleic acids of interest.

In vitro polymerization techniques have enormously benefitted the fields of biotechnology and medicine. The ability to manipulate nucleic acids with polymerization 1 reactions greatly facilitates techniques ranging from gene characterization and molecular cloning (including, but not limited to sequencing, mutagenesis, synthesis, and amplification of DNA), determining allelic variations, and detecting and screening of various diseases and conditions (e.g., hepatitis B).

An in vitro polymerization technique of great interest is the polymerase chain reaction (PCR). This method rapidly and exponentially replicates and amplifies nucleic acids of interest. PCR is performed by repeated cycles of denaturing a DNA template, usually by high temperatures, then annealing opposing primers to their complementary DNA strands, and then extending the annealed primers with a DNA polymerase. Multiple cycles of PCR result in an exponential amplification of the DNA template.

Unfortunately, PCR has limitations. These limitations range from 1) the rate of nucleotide incorporation, 2) the fidelity of nucleotide incorporation, 3) limitations on the length of the molecule to be amplified, and 4) the specificity of the polymerase.

Various methods to improve PCR exist. One approach is to optimize the reaction conditions, e.g., the reaction buffer, dNTP concentrations, or reaction temperatures. Another approach is to add various chemical compounds, e.g., formamide (Sarkar, G., et al. Nucl. Acids Res. 18: 7465, 1990), tetramethylammonium chloride (TMAC), or dimethyl sulfoxide (DMSO; Chevet et al., Nucl. Acids Res. 23:3343-3344, 1995; Hung et al., Nucl. Acids Res. 18:4953, 1990) to increase the specificity and/or the yield of the PCR reaction.

Other attempts include adding various proteins, such as replication accessory factors. Replication accessory factors known to be involved in DNA replication have also increased yields and the specificity of PCR products. For example, E. coli single-stranded DNA binding protein, such as SSB, has been used to increase the yield and specificity of primer extension and PCR reactions (U.S. Pat. Nos. 5,449,603, and 5,534,407). Another protein, the gene 32 protein of phage T4, appears to improve the ability to amplify larger DNA fragments (Schwartz et al., Nucl. Acids Res. 18:1079,

An important modification that has enhanced the ease and specificity of PCR is the use of Thermus aquaticus DNA polymerase (Taq) in place of the Klenow fragment of E. coli DNA pol I (Saiki et al., Science 230: 1350-1354, 1988). The use of Taq obviates any need for repeated enzyme additions, permits elevated annealing and primer extension temperatures, and enhances specificity. Further, this modification has enhanced the specificity of binding between the primer and its template. But, Taq has a drawback because it does not have 3′ to 5′ exonuclease (proofreading) activity and, therefore, cannot excise incorrect nucleotides added to the ends of the amplified products. Due to this limitation, the fidelity of Taq-PCR reactions typically have suffered. Therefore, those in the field have searched for alternative thermostable polymerases with proofreading activity.

Polymerases having proofreading activity have been found in archaea (archaebacteria). Archaea is a third kingdom, different from either the eukaryotes or the eubacteria. Many archaea are thermophilic bacteria-like organisms that can grow in extremely high temperatures, i.e., 100° C. One such archaea is Pyrococcus futiosus. A monomeric polymerase from Pyrococcus furiosus, referred to as Pfu polymerase or Pfu, has been identified that has the desired proofreading activity and synthesizes nucleic acids of interest at high temperatures (Lundberg et al., Gene 108: 1-6, 1991; Cline et al., Nucl. Acids Res. 24: 3546-3551, 1996). A second DNA polymerase has been identified in P. furiosus which has two-subunits (DP1/DP2) and is referred to as P. furiosus pol II. (European Patent Application EP0870832, Kato et al., published Oct. 14, 1998; Uemori et al., Genes to Cells, 2:499-512, 1997). These polymerases may also be enhanced by accessory factors.

Certain natural proteins exist in archaea, i.e., PEF (polymerase enhancing factors) that exhibit deoxyuracil triphosphatase (dUTPase) activity and that enhance the activity of Pfu (published PCT Application No. WO 98/42860, Hogrefe et al., published on Oct. 1, 1998). The presence of deoxyuracil-containing DNA in a DNA polymerization reaction inhibits polymerase activity (Lasken et al., J. Biol. Chem. 271: 17692-17696, 1996). Specifically, during the course of a normal PCR reaction, a dCTP may be deaminated into dUTP, thereby introducing a deoxyuridine into the newly synthesized DNA (dU-DNA). When this newly synthesized DNA is thereafter amplified, the presence of the deoxyuridine inhibits Pfu. dUTP may also be present in some commercial dNTPs preparations. PEF substantially prevents dUTP incorporation and, thus, substantially avoids the inhibition of Pfu. Accordingly, PEF optimizes the activity of Pfu.

Another method for improving PCR is to use DNA polymerase blends which provide longer target-length capability, higher product yields, greater sensitivity, and faster cycling times than can be achieved with single enzyme formulations. Certain commercial blends presently available consist predominantly of Taq (or a related non-proofreading DNA polymerase), blended with a small amount of a proofreading enzyme such as Pfu. Two commercial DNA polymerase blends, marketed by Stratagene, are TaqPlus Long and TaqPlus Precision.

The TaqPlus Long PCR System, employing a mixture of Taq and Pfu and using 2 different buffers: low-salt (for 0-10 kb templates) and high-salt (for 10-18.5 kb templates), is suitable for amplifying genomic targets up to 18.5 kilobases (kb) and cloned targets up to 35 kb in length. The TaqPlus Precision PCR System was specifically formulated to provide high product yield and fidelity.

Another commercially available DNA polymerase blend is used in the Expand™ 20 kb^{plus} PCR System (Boehringer Mannheim). According to its manufacturer, it can amplify 20-40 kb genomic targets and lambda targets of greater than 40 kb. The Expand™ 20 kb^{plus} polymerase is a blend of Taq and Pwo (proofreading component) DNA polymerases.

Several mechanisms have been proposed to explain the role of the minor proofreading component in the commercial DNA polymerase blends for amplifying long DNA targets (“long PCR” blends). One explanation, proposed by Barnes, is that Pfu acts to correct mismatches that Taq cannot efficiently extend (Barnes, Proc. Natl. Acad. Sci. 91:2216-20, 1994). Such a proofreading role is supported by the observation that Pfu that substantially lacks 3′-5′ exonuclease activity (exo⁻ Pfu) typically cannot be used in place of Pfu in polymerase blends to amplify long targets.

Additionally, the use of two DNA polymerases may overcome limitations that arise due to one enzyme's inability to incorporate opposite abasic sites (or other lesions) or to carry out processive synthesis through regions of template secondary structure (Eckert and Kunkel, J. Biol. Chem. 268:13462, 1993). The DNA polymerase blends described by Barnes contained no more than 20% Pfu (U/U), and optimal performance was achieved with blends consisting of 0.1-1% Pfu (Barnes, Proc. Natl. Acad. Sci. 91:2216-20, 1994; U.S. Pat. No. 5,436,149). Blends containing a higher proportion of Pfu exhibited reduced product yield and target-length capability, which Barnes attributed to the presence of excess 3′-5′ exonuclease activity.

The efficiency of Pfu-catalyzed PCR reactions can be limited by the accumulation of dUTP during temperature cycling, and its subsequent incorporation into PCR products. Improvements in product yield and target-length capability of Pfu-catalyzed reactions can be achieved with the addition of a thermostable dUTPase, such as PEF, which prevents dUTP accumulation and incorporation into DNA (published PCT Application No. WO 98/42860, Hogrefe et al.). PCRs carried out with P. furiosus pol II are also enhanced by PEF.

Due to the problems encountered with certain presently available DNA polymerase blends (high non-proofreading component: low proofreading component), they typically are unable to efficiently amplify complex DNA templates longer than about 20 kb with high fidelity. There is a need for more advantageous thermostable DNA polymerase blends and novel compositions to provide high-fidelity amplification of longer DNA templates. There is also a need in the art for a universal reaction mixture capable of supporting synthesis, amplification, or mutagenisis of a broad range of DNA templates. The present invention addresses these needs.

According to certain embodiments, the invention provides compositions comprising: (a) a thermostable non-proofreading DNA polymerase, (b) a thermostable proofreading DNA polymerase, and (c) a factor that substantially inhibits the incorporation of undesired nucleotides or analogs thereof into a DNA polymer.

In certain embodiments, the invention provides compositions or buffers that enhance a polymerization reaction involving DNA polymerases.

In certain embodiments, the invention provides compositions wherein the amount of the proofreading DNA polymerase is greater than, or less than or about equal to, the amount of non-proofreading DNA polymerase, as determined by units of polymerase activity.

In certain embodiments, the factor that substantially inhibits the incorporation of undesired nucleotides or analogs is a dUTPase, for example a thermostable dUTPase. In certain embodiments, the thermostable dUTPase is PEF, SIRV dUTPase (Prangishvili et al., J. Biol. Chem. 273:6024-9, 1998), a thermostable archaeal dUTPase, a dUTPase from a thermophilic or hyperthermophilic eubacteria, or a dUTPase from a mesophilic organism.

In certain embodiments, the invention provides compositions further comprising one or more of the following additional components: PCR additives, including, but not limited to, betaine, glycerol, TMAC, polyethylene glycol (PEG), DMSO, gelatin, and/or non-ionic detergents; dNTP analogs, including, but not limited to, 7-deaza-2′-deoxyguanosine triphosphate, that destabilize DNA secondary structure; enzymes, including, but not limited to, enzymes such as pyrophosphatases or ligases; and replication accessory factors including, but not limited to, archaeal PCNA, archaeal RFC-P38, RFC-P55, archaeal RFA, and/or archaeal helicases, e.g., dna2 and helicases 2 to 8 (U.S. Provisional Patent Application No. 60/146,580). PCNA is a “sliding clamp” protein that stabilizes the interaction between the polymerase and the primed single-stranded DNA template and enhances synthesis of long DNA strands (also known as “processivity”) (Baker and Bell, Cell 92: 295-305, 1998). RFC is a “clamp-loading” protein complex that assembles the PCNA protein.

In certain embodiments, the compositions comprise an archaeal polymerase as the proofreading DNA polymerase. In certain embodiments, the non-proofreading DNA polymerase comprises Taq or a thermostable eubacterial polymerase.

In certain embodiments, the proofreading archaeal polymerase is Pfu or related family B or α-type polymerases found in other members of the archaea.

In certain embodiments, the proofreading archaeal polymerase is P. furiosus pol II polymerase or related enzymes found in other members of the archaeal DNA polymerase II family.

In certain embodiments of the invention, the compositions comprise a proofreading archaeal DNA polymerase and a non-proofreading DNA polymerase, which may include either an eubacterial DNA polymerase or, alternatively, an archaeal DNA polymerase that substantially lacks 3′-5′ exonuclease activity, either naturally, or due to mutagenesis or modification.

In certain embodiments, the composition comprises Pfu as the proofreading DNA polymerase and Taq as the non-proofreading DNA polymerase. In certain embodiments, the Pfu:Taq ratio varies from about 1:1 to about 2 to 3:1, respectively, or even higher (i.e., greater than 3:1 Pfu:Taq). In certain embodiments the Pfu:Taq ratio is less than about 1:1, for example, but not limited to, Pfu:Taq ratios of about 1:1.01 to about 1:8.

In certain embodiments, the archael polymerase is related to Pfu or P. furiosus pol II. In certain embodiments, the archaeal polymerase is KOD (Toyobo), Pfx (Life Technologies Inc.), Vent (New England Biolabs), Deep Vent (New England Biolabs), Pwo (Roche Molecular Biochemicals), or JDF3 (Thermococcus sp. JDF3).

In certain embodiments, the invention provides methods for amplifying, synthesizing (including replicating), and mutagenizing nucleic acids of interest comprising using the novel compositions and buffers disclosed herein.

According to certain embodiments, kits are provided for amplifying, synthesizing, or mutagenizing nucleic acids of interest, comprising a non-proofreading DNA polymerase, a proofreading DNA polymerases, and a factor that substantially inhibits the incorporation of undesired nucleotides or analogs thereof into a DNA polymer. In certain embodiments, these kits further comprise a buffer that enhances a polymerization reaction involving the DNA polymerases and wherein the amount of proofreading DNA polymerase is greater than the amount of non-proofreading DNA polymerase, as determined by units of polymerase activity. In certain embodiments, kits are provided wherein the amount of the proofreading polymerase is less than or about equal to the amount of the non-proofreading polymerase, as determined by units of polymerase activity. It is understood by persons of skill in the art that the components of such kits may be provided either individually, i.e., in separate containers, or with at least two components combined prior to use in an amplification, synthesis, or mutagenizing reaction.

### BRIEF DESCRIPTION OF THE DRAWINGS

(All ratios are determined by units of polymerase activity.)

FIG. 1 illustrates PCR amplification of a 23 kb β-globin target in reaction buffer optimized for Pfu (50 mM Tricine, pH 9.1, 8 mM (NH₄)₂SO₄, 0.1% Tween 20, 2.3 mM MgCl₂ and 75 μg/ml nuclease-free bovine serum albumin (BSA)) using various Taq:Pfu DNA polymerase blend ratios, both with (lanes 1-4) and without PEF (lanes 5-8; 1 U/50 μl reaction). DNA polymerase blends used were 1.3:1 Taq:Pfu (lanes 1, 5), 1:1.25 Taq:Pfu (lanes 2,6), 1:1.8 Taq:Pfu (lanes 3,7), and 1:2.5 Taq:Pfu (lanes 4, 8). Lane M contains the LTI 5 kb marker.

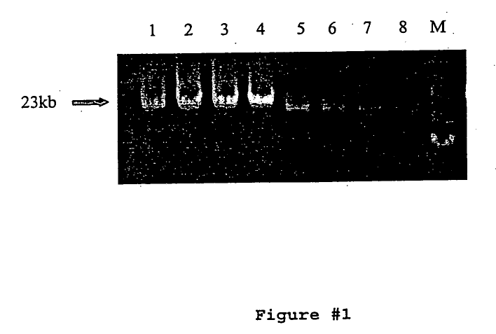

FIG. 2 illustrates PCR amplifications comparing TaqPlus Long supplemented with 1 U/reaction PEF to a novel composition comprising a DNA polymerase blend of Pfu:Taq at a 2:1 ratio and PEF. All PCRs were carried out in a novel buffer (50 mM Tricine, pH 9.1, 8 mM (NH₄)₂SO₄, 0.1% Tween 20, 2.3 mM MgCl₂, and 75 μg/ml nuclease-free BSA). TaqPlus Long with PEF was used in lanes 1-3 and the novel composition was used in lanes 4-6. A human genomic DNA template was used at 240 ng (lanes 1, 4), 480 ng (lanes 2, 5), and 720 ng (lanes 3, 6). Lane M contains the LTI 5 kb marker.

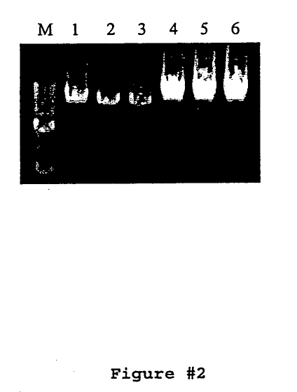

FIG. 3 illustrates PCR amplification showing optimization of the ammonium sulfate (panel A) and DTT (panel B) concentrations in a preferred buffer (50 mM Tricine, pH 9.1, 0.1% Tween-20, 2.3 mM MgCl₂, and 75 μg/ml nuclease-free BSA). The following β-globin targets were amplified with 5U of the novel blend of 2:1 Pfu:Taq plus 1 U PEF, in the presence of various concentrations of ammonium sulfate: (A) 23 kb template (1) 8 mM, (2) 6 mM, (3) 4 mM, (4) 2 mM; 19 kb template (5) 8 mM, (6) 6 mM, (7) 4 mM, (8) 2 mM and (9) 2 mM. Lane M contains the LTI 5 kb marker. The following β-globin targets were amplified in the presence of various concentrations of DTT: (B) 23 kb template in 8 mM ammonium sulfate (1) 0 mM DTT, (2) 1 mM DTT, (3) 2 mM DTT, (4) 4 mM DTT, (5) 6 mM DTT; 23 kb template in 6 mM ammonium sulfate (6) 0 mM DTT, (7) 1 mM DTT, (8) 2 mM DTT, (9) 4 mM DTT, and (10) 6 mM DTT.

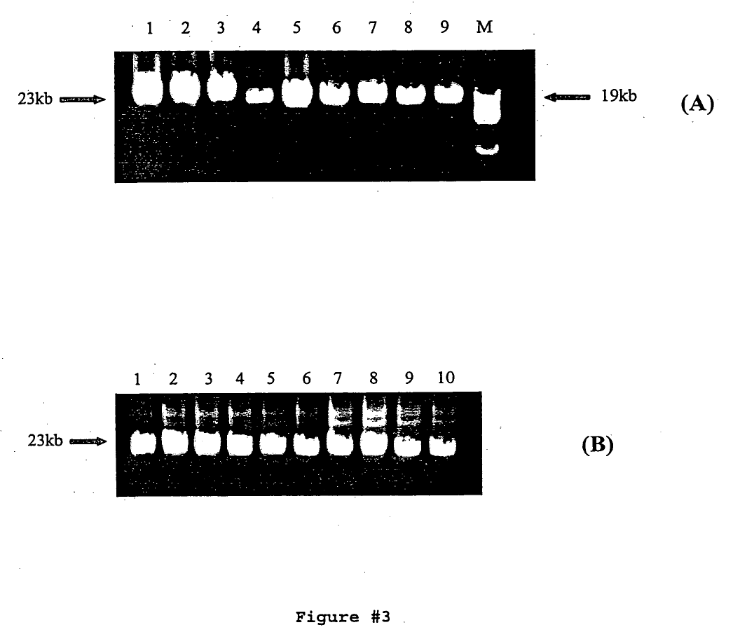

FIG. 4 illustrates PCR amplification showing a comparison of PCR amplification using TaqPlus Long with its optimal buffer (20 mM Tris HCl, pH 9.2, 60 mM KCl, and 2 mM MgCl₂) compared with PCR amplification using novel DNA polymerase blends and a novel optimized buffer (50 mM Tricine, pH 9.1, 8 mM (NH₄)₂SO₄, 0.1. % Tween-20, 2.3 mM MgCl₂, 75 μg/ml nuclease-free BSA, and 2 mM DTT). Amplifications of the following β-globin targets were carried out: 17 kb (lane 1), 19 kb (lane 0.2), 23 kb (lanes 3, and 5-12), and 30 kb (lane 4). The polymerases and amounts used were: TaqPlus Long, 5U (lanes 1-4), a novel-(optimal) blend of 2:1 Pfu:Taq, 5U (lane 5), 4U (lane 6), 3U (lane 7), 2U (lane 8); novel blend of 3:1 Pfu:Taq, 5U (lane 9), 4U (lane 10), 3U (lane 11), 2U (lane 12). All reactions carried out with a 2:1 or 3:1 Pfu:Taq polymerase blend included 1 U/reaction of PEF (lanes 5-12). Lane M contains Lambda/Hind III marker.

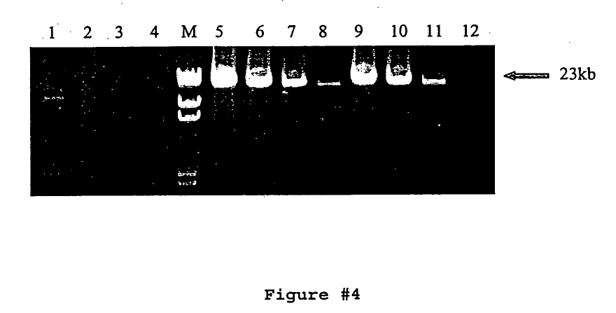

FIG. 5 illustrates PCR showing the effect of DMSO on amplifications of long genomic targets. In panel (A), amplifications of the 30 kb β-globin target were carried out in the presence of the following concentrations of DMSO: 0% (lane 1), 1% (lane 2), 2% (lane 3), or 3% (lane 4). In panel (B), the 26 kb β-globin target was amplified with the following concentrations of DMSO: 0% (lane 1) and 3% (lane 2). All PCR reactions were carried out using a 2:1 Pfu:Taq polymerase blend (5 U/reaction) and PEF (1 U/reaction) in a reaction buffer optimized for Pfu (50 mM Tricine, pH. 9.1, 8 mM (NH₄)₂SO₄, 0.1% Tween-20, 2.3 mM MgCl₂, 75 μg/ml nuclease-free BSA, and 2 mM DTT). Lane M contains Lambda/Hind III marker.

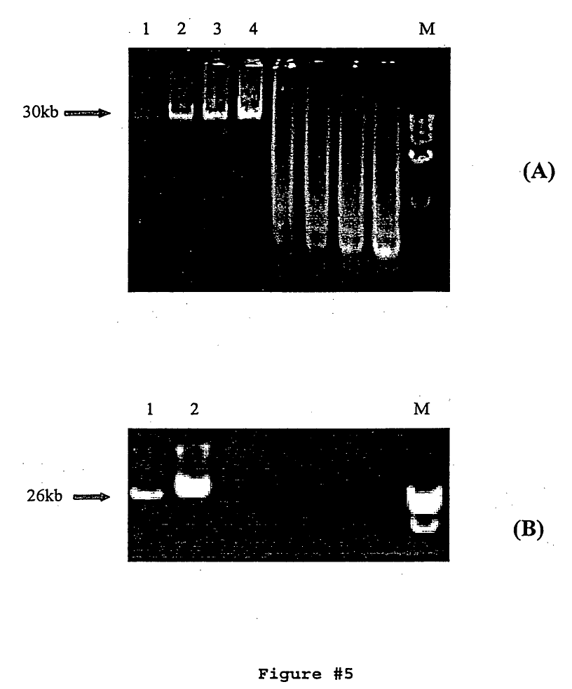

FIG. 6 shows PCR amplifications of short genomic targets using the novel 2:1 Pfu:Taq blend with a novel buffer optimized for this blend (50 mM Tricine, pH 9.1, 8 mM (NH₄)₂SO₄, 0.1% Tween-20, 2.3 mM MgCl₂, 75 μg/ml nuclease-free BSA, and 2 mM DTT) or TaqPlus Long PCR with low salt buffer (20 mM Tris-HCl, pH 8.8, 10 mM KCl, 10 mM (NH₄)₂SO₄, 2 mM MgSO₄, 0.1% Triton X-100, and 100 μg/ml nuclease-free BSA). The following portions of human α1 anti-trypsin gene were amplified: 4 kb (panel A), 900 bp (panel B), and 105 bp (panel C). In panels A and B, PCRs were carried out with 2.5 U/reaction of TaqPlus Long (Taq:Pfu 16U:1U; lanes 1 and 2) or 2.5 U/reaction of the novel Pfu:Taq 2:1 blend with 1 U/reaction of PEF (lanes 3 or 4). In panel C, PCRs were carried out the novel Pfu:Taq 2:1 blend in the presence of 3% DMSO (lanes 1 and 2), 4% DMSO (lane 3), 5% DMSO (lane 4), and 0% DMSO (lanes 5-8). Lane M contains the Hinfl marker.

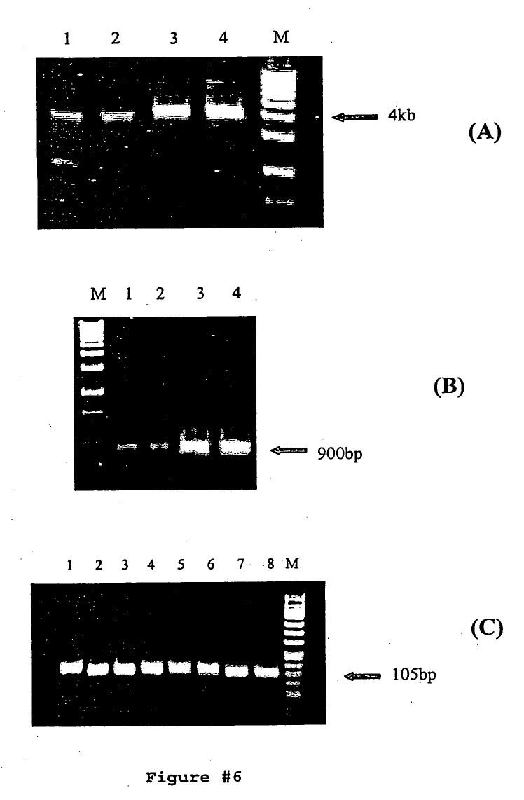

### DETAILED DESCRIPTION OF THE INVENTION

The following description should not be construed to limit the scope of this invention to any specifically described embodiment. Various aspects and embodiments of this invention will be apparent from the disclosure as a whole in context with the knowledge of one skilled in the art. In addition, the description herein, in combination with information known or available to persons of ordinary skill in the art, enables the practice of the subject matter encompassed by the following claims.

The invention provides novel compositions, comprising a thermostable non-proofreading DNA polymerase, a thermostable proofreading DNA polymerase, and a factor that substantially inhibits the incorporation of undesired nucleotides or analogs thereof into a DNA polymer, for amplifying a broad range of amplicon sizes, for example, but not limited to, genomic targets of 0.1 to 37 kb and up to 45 kb long for less complex lambda DNA targets. In certain embodiments, the compositions further comprise a buffer that enhances a polymerization reaction involving the DNA polymerases.

The term “substantially inhibits the incorporation of undesired nucleotides or analogs” means suppression of the incorporation of the undesired nucleotides or analogs to the point where the presence of incorporated undesired nucleotides or analogs does not materially affect the properties or yield of the polymerization reaction. For example, a dUTPase cleaves the dUTP molecule such that it is not incorporated into the DNA polymer. Proofreading DNA polymerases, in contrast, excise incorrect nucleotides or analogs that have already been incorporated into the DNA polymer. The term nucleotide “analog” refers to a molecule that is recognized by a polymerase and incorporated into the DNA polymer, excluding the dNTP that corresponds to the matching base on the template strand, for example A:T, G:C.

The terms “enhances a polymerization reaction” or “enhances a polymerization reaction yield” are intended to mean increasing the fidelity, increasing specificity, increasing synthesis through regions of template secondary structure, increasing the synthesis of long products, increasing the processivity, increasing the product yield and/or rate of nucleotide incorporation into a DNA polymer, compared to a polymerization reaction involving the DNA polymerases in a reaction buffer consisting of 20 mM Tris HCl, pH 9.2, 60 mM KCl, and 2 mM MgCl₂. The polymerization reaction may be enhanced, for example, by increasing the activity of the proofreading DNA polymerase, the non-proofreading DNA polymerase, or the factor that substantially inhibits incorporation of undesired nucleotides or analogs, either individually or in combination. The polymerization reaction may also be enhanced, for example, by increasing the activity of a replication accessory factor or other component of the inventive compositions or methods.

The use of “hot start” technology is within the scope of the claims. Hot start technology includes, but is not limited to, the use of reversibly-inactivated polymerases. Reversibly-inactivated DNA polymerases have been generated by either chemical or immunological inactivation. Immunological inactivation may result, for example, from combining neutralizing antibodies with a DNA polymerase. An example of a reversible chemical inactivation of DNA polymerases is described in Example 6 below.

Biological activity of the reversibly-inactivated polymerases can be recovered, e.g., by heating the chemically or immunologically inactivated component. Heating at appropriate temperatures can cause either the inactivating antibody molecules to be released or the chemical modification to be removed, resulting in the return of biological activity (Kellogg et al, BioTechniques 16:1134-37, 1994; Nieto et al., Biochim. Biophys. Acta 749:204-10, 1983). Commercial formulations of Taq with hot start capability, e.g., AmpliTaq Gold (Perkin Elmer) and Platinum Taq (Life Technology) are available. Other methods to reactivate reversibly-inactivated proteins include pH shift or other triggerable release mechanisms. It is expected that this reversible-inactivation technology will be applicable to numerous proteins, including, but not limited to, dUTPases, helicases, accessory factor proteins, various enzymes, etc.

According to certain embodiments, the invention provides compositions further comprising a thermostable proofreading DNA polymerase in a greater amount than the amount of thermostable non-proofreading DNA polymerase, as determined by units of polymerase activity. Also provided are compositions wherein the amount of the thermostable proofreading DNA polymerase is less than or about equal to the amount of the thermostable non-proofreading DNA polymerase, as determined by units of polymerase activity.

Certain embodiments of the invention provide compositions wherein the amount of thermostable proofreading DNA polymerase is greater than the amount of thermostable non-proofreading DNA polymerase, as determined by units of polymerase activity, and further comprising a buffer that enhances the polymerization reaction.

The terms “amplifying” or “amplification” refers to both linear and exponential amplification. Linear amplification includes, but is not limited to, non-cyclic primer extension. Examples of exponential amplification include, but are not limited to, polymerase chain reaction, rolling circle amplification, and strand displacement amplification.

The term “polymerase” as used herein is intended to encompass naturally-occurring or native polymerase, recombinantly-derived polymerase, as well as truncations, deletions, deriviatives, and variations of native or recombinantly-derived polymerase.

The term “units of polymerase activity” means the activity required to incorporate dNTPs into a template at the optimal temperature for that polymerase. One unit of polymerase activity is defined as the amount of polymerase needed to incorporate 10 nmoles of dNTPs in 30 minutes at the optimal temperature for that polymerase. Determining the optimal temperature for polymerase activity may require running a series of experiments in which all of the experimental conditions are held constant except reaction temperature, which is varied through a suitable range of temperatures. The optimal temperature would be that temperature at which maximum polymerization activity occurs. The optimal temperature for Pfu and Taq is 72° C.

For the purposes of determining the unit concentration of a polymerase used in PCR, polymerase samples are titrated in a series of PCR reactions. Unit concentrations are assigned based upon PCR performance relative to previously qualified lots of the same PCR enzyme (e.g., Pfu, Taq) (See Example 5).

When used in referring to dUTPases in general, or to PEF specifically, “units” means dUTPase units. One dUTPase unit is defined as the amount of enzyme which produces 1.757 nmole of inorganic pyrophosphate (“PPi”) per hour. The dUTPase assay (dUTP converted to dUMP+PPi) is carried out using the Sigma pyrophosphate reagent #P7275. The dUTPase, such as PEF, is incubated with 10 mM solutions of dUTP in 1× cloned Pfu buffer (20 mM Tris, pH 8.8, 10 mM KCl, 10 mM (NH₄)₂SO₄, 2 mM MgSO₄, 0.1% Triton X-100, 100 μg/ml nuclease-free BSA) for 1 hour at 85° C. PPi production is quantified as described in Sigma's Technical bulletin (Sigma Technical Bulletin No. BI-100, February 1999).

The term “hyperthermophilic” refers to organisms that grow optimally at temperatures of approximately 80° C.-85° C. and higher. The term “thermophilic” refers to organisms that grow optimally at temperatures of approximately 55° C.-85° C. The term “mesophilic” refers to organisms that grow at temperatures between approximately 15° C. and 45° C.

The term “thermostable,” when referring to a polymerase or a dUTPase, means an enzyme which is stable and active at temperatures from about 50-99° C. In certain embodiments, enzymes are stable at temperatures up to at least 90-99° C., such that the enzyme retains activity during temperature cycling, including at denaturation temperatures. In certain embodiments, thermostable enzymes will retain biological activity at temperatures between 68 and 95° C., the respective primer extension and template denaturation temperatures commonly used.

In certain embodiments, the novel compositions comprise an archaeal polymerase as the proofreading DNA polymerase. Thermostable archaeal DNA polymerase may be obtained from archaea such as Pyrococcus furiosus (Stratagene), Pyrococcus species GB-D (Deep Vent, New England Biolabs), Pyrococcus species strain KOD1 (Tagaki et al., Appl. Environ. Microbiol. 63:4504-10, 1997) (KOD, Toyobo; Pfx, Life Technologies), Pyrococcus woeseii (Pwo, Roche Molecular), Pyrococcus horikoshii, Pyrodictium occultum, Archaeoglobus fulgidus, Sulfolobus solfataricus, Sulfolobus acidocaldarius, Thermococcus litoralis (Vent, New England Biolabs), Thermococcus sp. 9 degrees North-7 (Southworth et al., Proc. Natl. Acad. Sci. 93:5281-5, 1996), Thermococcus gorgonarius, Methanobacterium thernoautotrophicum, Methanococcus jannaschii, and Thermoplasma acidophilum, available from the American Type Culture Collection or the DSMZ German collection of Microorganisms and Cell Cultures. Related archaea from which the thermostable archaeal DNA polymerase may be obtained have been described (Archaea: A Laboratory Manual, Robb, F. T and Place, A. R., eds, Cold Spring Harbor Laboratory Press, 199.5).

In certain embodiments, the non-proofreading DNA polymerase comprises a thermostable eubacterial polymerase. Thermostable eubacterial polymerases have been described in Thermus species (aquaticus, flavus, thermohilus HB-8, ruber, brokianus, caldophius GK14, filiformis), and Bacillus species (stearothermophilus, caldotenex YT-G, caldovelox YT-F). Commercial enzymes that are related to eubacterial pol I enzymes include Taq (Stratagene) Tth (Perkin Elmer), Hot Tub/Tfl (Amersham), Klen Taq (ClonTech), Stoffel fragment (Perkin Elmer), DynaZyme (Finnzymes), Bst (New England Biolabs), and Bca (Panvera). Thermostable pol III DNA polymerases have been described in Thermus aquaticus (Huang, et al. J. Mol. Evol. 48:756-69, 1999) and Thermus thermohilus (McHenry et al., J. Mol. Biol. 272:178-89, 1997), but could be obtained from other thermophilic eubacteria. Additional thermophilic eubacteria have been described (Kristjansson, J. K., Thermophilic Bacteria, CRC Press, Inc., 1992). Certain eubacterial pol I polymerases possess 3′-5′ exonuclease activity such as Thermotoga maritima pol 1. Non-proofreading versions of such eubacterial polymerases could be used as the non-proofreading component in this invention. 3′-5′ exonuclease-minus versions (exo⁻) can be prepared by methods well-known in the art (Derbyshire et al., Methods of Enzymology 262:3-13, 1995).

In certain embodiments, the proofreading archaeal polymerase is P. furiosus pol II polymerase or homologous enzymes found in other members of the archaea. Archaea which contain genes that exhibit DNA sequence homology to P. furiosus pol II subunits have been described (Makinjemi, M. et al., Trends in Biochem. Sci. 24:14-16, 1999; Ishino et al., J. Bacteriol., 180:2232-6, 1998).

In certain embodiments, the factor that substantially inhibits the incorporation of undesired nucleotides or analogs thereof into a DNA-polymer is a dUTPase. In certain embodiments, the factor is a thermostable dUTPase. In certain embodiments, the factor is a thermostable archaeal dUTPase. In certain embodiments, the factor is the archaeal dUTPase PEF. PEF can enhance several archaeal DNA polymerases, including Pwo from Pyrococcus woeseii, Deep Vent from Pyrbcoccus sp. GB-D, KOD from Thermococcus sp. KOD, Vent from Thermococcus litoralis and JDF-3 from Thermococcus sp. JDF-3. In contrast with the archaeal polymerases, PEF does not enhance the activity of Taq or other DNA polymerases of eubacterial origin. A broad range of PEF concentrations enhance PCRs carried out with DNA polymerase blends. A thermostable dUTPase from any thermophilic or hyperthermophilic eubacteria or archaea, or SIRV dUTPase, may enhance DNA polymerase blends containing an archaeal DNA polymerase as the proofreading component.

Polymerization reaction yields are increased when PEF is included with DNA polymerase blends containing archaeal polymerases. The activity and fidelity of these polymerase blends can also be increased by several methods, including increasing the proportion of the archaeal proofreading component in the blend, enhancing the activity of the archaeal proofreading polymerase by the addition of replication accessory factors, PCR additives, and optimizing the reaction buffer to enhance the polymerization reaction.

Various reaction buffers have been developed for use in long PCR reactions (i.e., amplification of templates of 20 kb or longer) using DNA polymerase blends that contain predominantly either the Taq or Tth eubacterial polymerases. Tris- or tricine-based reaction buffers (20-50 mM) are used, which exhibit a higher pH, i.e., 8.7-9.2 at room temperature, than standard PCR reaction buffers, and generally have a room temperature pH of 8.3-8.4. Buffers with higher pH have been proposed to enhance long PCR reactions by facilitating template denaturation and reducing DNA damage (Barnes, Proc. Natl. Acad. Sci. 91:2216-20, 1994; Cheng et al., Proc. Natl. Acad. Sci. 91:5659, 1994).

In addition, certain PCR additives that facilitate standard PCR have also been shown to enhance long PCR reactions, including DMSO (1-5%; Cheng et al., Proc. Natl. Acad. Sci. 91:5659, 1994), glycerol (5-8%; Cheng et al., Proc. Natl. Acad. Sci. 91:5659, 1994; Foord et al., in PCR Primer, eds. Dieffenbach and Dveskler, Cold Spring Harbor Laboratory Press, 1995), gelatin (0.01%; Foord et al., in PCR Primer, eds. Dieffenbach and Dveskler, Cold Spring Harbor Laboratory Press, 1995), and Tween 20 (0.05%; Foord et al., in PCR Primer, eds. Dieffenbach and Dveskler, Cold Spring Harbor Laboratory Press, 1995).

Potassium salts are commonly used in standard Taq- or Tth-based PCR reaction buffers at concentrations of 50 or 100 mM KCl, respectively. Cheng has reported, however, that lowering the potassium concentration (KCl, KOAc) by 10-40% is more favorable for long PCR reactions using Taq- or Tth-based blends, although reduced specificity was noted (Cheng et al., Proc. Natl. Acad. Sci. 91:5659, 1994). Barnes has developed reaction buffers for KlenTaq-based polymerase blends (ClonTech), that used ammonium sulfate but not potassium (Barnes, Proc. Natl. Acad. Sci. 91:2216, 1994). As in standard PCR, Mg²⁺ is used for polymerase activity, and optimal concentrations used in long PCR reactions have ranged from 1.0-3.5 mM MgCl₂ or MgSO₄ (Barnes, Proc. Natl. Acad. Sci. 91:2216, 1994; Cheng et al., Proc. Natl. Acad. Sci. 91:5659, 1994; Foord et al., in PCR Primer, eds. Dieffenbach and Dveskler, Cold Spring Harbor Laboratory Press, 1995).

The reaction buffers developed for Taq- or Tth-based polymerase blends were found to be sub-optimal for enhancing blends containing Pfu and PEF. KCl was found to be inhibitory to Pfu and the addition of PEF did not overcome this inhibition. PEF enhancement was obtained when potassium salts were omitted from the reaction buffers.

According to certain embodiments of the invention, for Pfu-containing blends, a buffer is provided that contains 50 mM tricine, pH 9.1, 8 mM ammonium sulfate, 0.1% Tween 20, 2.3 mM MgCl₂, 75 μg/ml nuclease-free BSA, and 2 mM dithiothreitol (DTT). Certain components can be substituted for other reagents and the concentrations of some components can be varied, without substantially reducing performance in synthesis, mutagenizing, or amplification reactions.

In certain embodiments, the invention provides buffers and compositions comprising: about 20-70 mM Tricine or about 10-70 mM Tris-HCl; 0 to about 16 mM ammonium sulfate; about 0.01-0.2% Tween-20 or Triton X-100; between about 1.5 mM and 3 mM MgCl₂, MgSO₄, or C₄H₆O₄Mg; 0 to about 100 μg/ml nuclease-free BSA; and 0 to about 4 mM DTT. The preferred pH is between about 8.0 to about 9.5, more preferably between 8.4 to 9.2, and most preferably the pH is 9.1.

The addition of DMSO was also found to improve amplification of extra long nucleic acid targets using Taq/Pfu polymerase blends containing PEF. Concentrations of about 3-7% DMSO were optimal, but the optimal concentration was found to vary from system-to-system and with nucleic acid target size. DMSO concentrations of up to about 10% are believed to be useful in improving amplification of extra long templates. Similar organic compounds, including, but not limited to dimethylformamide (DMF), betaine, glycerol, or TMAC, could also be used to improve-amplification of long nucleic acid targets (Landre et al., in PCR Strategies, M. Innis et al., eds., Academic Press, 1995; Henke et al., Nucleic Acids Research 25: 3957-3958, 1997).

It is expected that PEF will enhance DNA polymerase blends containing any archaeal polymerase as the proofreading component. The archaeal DNA polymerases Pwo, KOD, Vent, Deep Vent, JDF-3, and P. furiosus pol II are stimulated by PEF. Each of these enzymes may exhibit optimal activity in PCR reaction buffers containing different components and/or component concentrations than those found in the optimal Pfu reaction buffer. Using the buffer optimization procedures described herein, the skilled artisan will appreciate that optimal buffers for various thermostable proofreading DNA polymerases can be readily determined. Factors such as, but not limited to, pH, potassium ion concentration, ammonium sulfate concentration, reducing agents, stabilizing agents (e.g., but not limited to, proline, trehalose), and other buffer components, such as, but not limited to MOPS, HEPES, PIPES, may be important in optimizing a buffer to enhance polymerization reaction yield.

Although the DNA polymerase blends analyzed contained Taq as the non-proofreading component, other thermostable DNA polymerases substantially lacking 3′-5′ exonuclease activity may be utilized in PEF-containing blends. For example, PEF and a thermostable proofreading archaeal polymerase could be combined with any of a number of thermostable eubacterial polymerases. In addition to eubacterial DNA polymerases, the non-proofreading component may be an archaeal DNA polymerase that has been modified or mutated to eliminate the proofreading function. It has been previously demonstrated that an exo-Pfu/exo'Pfu blend could perform in long PCR, although typically not as well as KlenTaq I/Pfu polymerase blends (Barnes, Proc. Natl. Acad. Sci. 91:2216, 1996). Methods for preparing archaeal DNA polymerase mutants, with reduced or abolished proofreading activity, are well known in the art (See, e.g., Perler et al., Adv. Prot. Chem. 48:377, 1996).

The present invention also contemplates the use of blends of blends of DNA polymerases. For example, a formulation of three or more DNA polymerases, including at least one proofreading DNA polymerase and at least one non-proofreading DNA polymerase. Also contemplated by the present invention is the use of one or more factors that substantially inhibits the incorporation of undesired nucleotides or analogs thereof into a DNA polymer. Thus, in certain embodiments, the composition comprises one or more proofreading DNA polymerase, one or more non-proofreading DNA polymerase, and/or one or more factor.

The inventive methods, buffers, and compositions disclosed herein are also expected to be useful for synthesizing, amplifying, and mutagenizing nucleic acids at ambient and physiological temperatures, e.g., between about 20° C. and about 40° C. These applications would involve the use of DNA polymerases from mesophilic organisms. For example, but not as a limitation, the compositions may comprise a mesophilic proofreading archaeal DNA polymerase, obtained from Methanococcus voltae, and a mesophilic non-proofreading eubacterial DNA polymerase, such as exo⁻ Klenow fragment (Stratagene). It is apparent that numerous mesophilic proofreading and non-proofreading DNA polymerases found in the archaea, the eubacteria, bacteriophage, eukaryotic viruses and/or the eukaryotes could be successfully employed.

Certain embodiments of the invention are described in the following examples. However, these examples are offered solely for the purpose of illustrating the invention, and do not limit the invention.

### EXAMPLES

Methods

1. PCR Reaction Enzymes.

PCRs were carried out with DNA polymerase blends by: 1) adding the appropriate amounts of Pfu (Stratagene) and Taq (Taq2000, Stratagene) separately to the PCR buffer or by 2) combining Pfu (2.5 U/μl) and Taq (5 U/μl) at the appropriate ratios, and then adding an aliquot of the blend to the PCR buffers. dUTPase (PEF) was added separately to PCR reactions or reaction mixes to give a final-concentration of 1 U/50 μl.

2. PCR Reaction Conditions.

PCR reactions were performed in 200 μl thin-walled PCR tubes using the appropriate PCR buffer. All targets ≧17 kb were amplified using 500 μM each dNTPs, 0-6% DMSO, 240 ng genomic DNA or 15-60 ng lambda DNA, and 4 ng/μl each primer in a 50 μl reaction volume. Water, buffer, dNTPs, primers, DNA, DMSO, Pfu:Taq (5U/reaction), and PEF (1U/reaction) were combined and gently mixed. Reactions were then overlaid with approximately 20 μl of mineral oil to prevent sample evaporation during prolonged cycling times. All targets ≦6 kb were amplified using 200 μM each dNTP, 0-3% DMSO, 100 ng genomic DNA or 15-60 ng plasmid DNA, and 2 ng/μl of each primer in a 50 μi reaction volume. All components were added and mixed as above, although no mineral oil was used.

3. Genomic DNA.

Three sources of genomic DNA were used:

A) Promega (catalogue # G304A)

B) ClonTech (catalogue # 6550-1)

C) DNA isolated from cultured HeLa cells using the RecoverEase DNA isolation kit (Stratagene).

Wild type lambda DNA from Stratagene was isolated from purified lambda phage by phenol extraction and dialyzed against 10 mM Tris-HCl (pH 8.0), 1 mm EDTA. All DNA stocks were diluted to working concentrations (100 ng/μl genomic and 5 ng/μl lambda DNA) with distilled water (except the ClonTech product which was provided at 100 ng/μl) and stored at 4° C. to eliminate shearing due to repeated freezing/thawing.

4. Cycling Conditions.

The thermal cyclers used were the PTC-200 DNA Engine (MJ Research), the Model 9600 thermal cycler (Perkin-Elmer Biosystems), and the RoboCycler Gradient 96 (Stratagene). PCR profiles for each machine are as follows:

Profile A. Model 9600 and PTC-200.

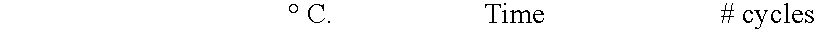

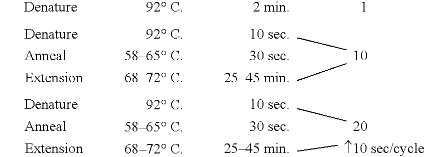

Profile B. Model 9600 and PTC-200.

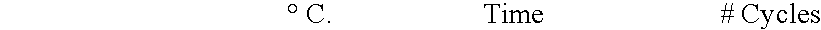

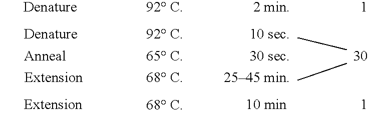

Profile C. RoboCycler Gradient 96.

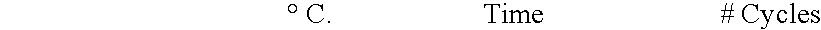

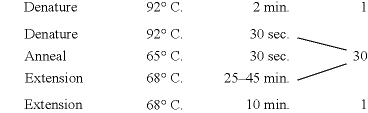

5. PCR Targets.

The following PCR targets were used to evaluate the performance of the amplification method using the novel 2:1 Pfu:Taq polymerase blend with PEF, in its novel buffer (50 mM Tricine, pH 9.1, 8 mM (NH₄)₂SO₄, 0.1% Tween-20, 2.3 mM MgCl₂, 75 μg/ml nuclease-free BSA, and 2 mM DTT):

1. A composition comprising: (a) a thermostable non-proofreading DNA polymerase, (b) a thermostable proofreading DNA polymerase, and (c) a factor that substantially inhibits the incorporation of undesired nucleotides or analogs thereof into a DNA polymer

2.-48. (canceled)


SEQUENCE LISTING (30 sequences)

SEQ ID NO: 1 - DNA, 30 bp, Artificial Sequence
  Description of Artificial Sequence PCR Primer
atgctattaaaaacatggacaacctcaagc

SEQ ID NO: 2 - DNA, 24 bp, Artificial Sequence
  Description of Artificial Sequence PCR Primer
cacaagggctactggttgccgatt

SEQ ID NO: 3 - DNA, 30 bp, Artificial Sequence
  Description of Artificial Sequence Primer
ctcagatatggccaaagatctatacacacc

SEQ ID NO: 4 - DNA, 30 bp, Artificial Sequence
  Description of Artificial Sequence PCR Primer
agcttcccaacgtgatcgcctttctcccat

SEQ ID NO: 5 - DNA, 30 bp, Artificial Sequence
  Description of Artificial Sequence PCR Primer
cagggcattgacagcagtcttctcctcagg

SEQ ID NO: 6 - DNA, 30 bp, Artificial Sequence
  Description of Artificial Sequence PCR Primer
tatggttatcaggaaacagtccaggatctc

SEQ ID NO: 7 - DNA, 30 bp, Artificial Sequence
  Description of Artificial Sequence PCR Primer
acagcaagaaagcgagcttagtgatacttg

SEQ ID NO: 8 - DNA, 34 bp, Artificial Sequence
  Description of Artificial Sequence PCR Primer
ccttcactgtctgcctaactccttcgtgtgttcc

SEQ ID NO: 9 - DNA, 34 bp, Artificial Sequence
  Description of Artificial Sequence PCR Primer
gcaggggtgctgcagaactctgagctgtacttcc

SEQ ID NO: 10 - DNA, 31 bp, Artificial Sequence
  Description of Artificial Sequence PCR Primer
tgtctccagcacacagcatgttgtcggtgac

SEQ ID NO: 11 - DNA, 22 bp, Artificial Sequence
  Description of Artificial Sequence PCR Primer
cagctggctgacattttcggtg

SEQ ID NO: 12 - DNA, 22 bp, Artificial Sequence
  Description of Artificial Sequence PCR Primer
ccgcctttacaatgtccccgac

SEQ ID NO: 13 - DNA, 21 bp, Artificial Sequence
  Description of Artificial Sequence PCR Primer
cctgaattttcggtgatgcct

SEQ ID NO: 14 - DNA, 23 bp, Artificial Sequence
  Description of Artificial Sequence PCR Primer
cctgttatcaagcactgcactgg

SEQ ID NO: 15 - DNA, 22 bp, Artificial Sequence
  Description of Artificial Sequence PCR Primer
gaatcagcgcacatggtacagc

SEQ ID NO: 16 - DNA, 20 bp, Artificial Sequence
  Description of Artificial Sequence PCR Primer
gcatcagtaagcgcattggc

SEQ ID NO: 17 - DNA, 21 bp, Artificial Sequence
  Description of Artificial Sequence PCR Primer
gtttgggttgtgctgttgctg

SEQ ID NO: 18 - DNA, 22 bp, Artificial Sequence
  Description of Artificial Sequence PCR Primer
cctgagggctcccagagagtgg

SEQ ID NO: 19 - DNA, 22 bp, Artificial Sequence
  Description of Artificial Sequence PCR Primer
ggtttagcacgaccacaacagc

SEQ ID NO: 20 - DNA, 23 bp, Artificial Sequence
  Description of Artificial Sequence PCR Primer
gaggagagcaggaaaggtggaac

SEQ ID NO: 21 - DNA, 23 bp, Artificial Sequence
  Description of Artificial Sequence PCR Primer
gaggtacagggttgaggctagtg

SEQ ID NO: 22 - DNA, 22 bp, Artificial Sequence
  Description of Artificial Sequence PCR Primer
gaaaataggagctcagctgcag

SEQ ID NO: 23 - DNA, 22 bp, Artificial Sequence
  Description of Artificial Sequence PCR Primer
ttggacagggatgaggaataac

SEQ ID NO: 24 - DNA, 32 bp, Artificial Sequence
  Description of Artificial Sequence PCR Primer
gagcaatggtcaaagtcaacgtcatccacagc

SEQ ID NO: 25 - DNA, 33 bp, Artificial Sequence
  Description of Artificial Sequence PCR Primer
catagcgaattcgcaaaacctttcgcggtatgg

SEQ ID NO: 26 - DNA, 33 bp, Artificial Sequence
  Description of Artificial Sequence PCR Primer
actacggaattccacggaaaatgccgctcatcc

SEQ ID NO: 27 - DNA, 18 bp, Artificial Sequence
  Description of Artificial Sequence PCR Primer
gacagtcactccggcccg

SEQ ID NO: 28 - DNA, 18 bp, Artificial Sequence
  Description of Artificial Sequence PCR Primer
cgacgactcgtggagccc

SEQ ID NO: 29 - DNA, 18 bp, Artificial Sequence
  Description of Artificial Sequence PCR Primer
gacagtcactccggcccg

SEQ ID NO: 30 - DNA, 18 bp, Artificial Sequence
  Description of Artificial Sequence PCR Primer
cgacgactcgtggagccc